(19) United States
(12) Patent Application Publication
(10) Pub. No.: US 2012/0191185 A1
(43) Pub. Date: Jul. 26, 2012

(54) OPHTHALMIC INTRAOCULAR LENS FOR PROMOTING HEALING

(76) Inventors: Micheal Colvin, Newburry Park, CA (US); Hossein Nafarieh, Fullerton, CA (US)

(21) Appl. No.: 13/358,108

(22) Filed: Jan. 25, 2012

Related U.S. Application Data
(60) Provisional application No. 61/436,436, filed on Jan. 26, 2011.

(51) Int. Cl. A61F 2/16

(52) U.S. Cl. 623/6.62

(57) ABSTRACT

An ophthalmic intraocular lens for promoting healing and inhibiting posterior chamber opacification includes an intraocular lens and a composition that is bonded to or coated on the intraocular lens. The composition includes one or more growth factors and one or more anti-mitosis drugs for inhibiting posterior chamber opacification.

This application claims the benefit under 35 U.S.C. Section 119(e) of U.S. Provisional application 61,436,436 filed Jan. 26, 2011.

### BACKGROUND OF THE DISCLOSURE

### Field of the Disclosure

The disclosure relates to drug and ophthalmic intraocular lens combinations and more particularly pertains to a new drug and ophthalmic intraocular lens combination for implantation in an aphakic or phakic eye where the natural lens has been removed due to damage or disease such as a cataractous lens or where an intraocular lens (IOL) is required for refractive correction. More specifically the disclosure relates to a combination of composition for enhancing postoperative healing and in particular for preventing posterior chamber opacification (PCO), also known as posterior capsule opacification.

In the extracapsular extraction method, the natural lens is removed from the capsular bag while leaving the posterior part of the capsular bag, and preferably at least part of the anterior part of the capsular bag, in place within the eye. The capsular bag remains anchored to the eye's ciliary body through the zonular fibers. The capsular bag thus provides a natural centering and locating means for the IOL within the eye. The capsular bag also continues its function of providing a natural barrier between the aqueous humor at the front of the eye and the vitreous humor at the rear of the eye. The bag and its surroundings are cleaned and polished to maximize the reduction of remaining epithelial cells. However, using the best known cleaning and polishing techniques, small amount of remaining epithelial cells can still proliferate in patients and often results in PCO. An extensive capsulorhexis contraction, including capsulorhexis occlusion postoperatively, can lead to IOL dislocation into the anterior chamber and pupillary block syndrome may take place in some eyes. Other complications may include retinal detachment wherein the capsular bag itself may dislocate due to weakness or breakage of the fibers, i.e. zonules, that hold it in place.

Opacification of the posterior capsule in the optical axis is therefore still a significant long-term complication ultimately affecting approximately 50% of the cases. After the cataract surgery, the remnant lens epithelial cells (LEC) can proliferate on the posterior lens capsule to form lens “fibers” and “bladder” cells (i.e. Elschnig's pearls) resulting in blocking of incoming light, that is PCO. PCO requires subsequent surgery which may include undesirable complications. It is therefore highly desirable to prevent posterior capsule opacification in the first place and thereby obviate the need for a subsequent posterior capsulotomy. Mechanical and pharmaceutical means have been utilized to reduce the onset of PCO, however, these have thus far not proven completely effective.

### SUMMARY OF THE DISCLOSURE

An embodiment of the disclosure meets the needs presented above by generally comprising an intraocular lens and a composition that is bonded to or coated on the intraocular lens. The composition includes one or more growth factors and one or more anti-mitosis drugs for inhibiting posterior chamber opacification.

The embodiment further includes the one or more growth factors and the one or more anti-mitosis drugs are covalently, non-covalently, or a combination thereof bound to the surface of the implant using wet chemical, photochemical, plasma, gamma irradiation or polymerization techniques.

The embodiment further includes the one or more growth factors and the one or more anti-mitosis drugs are bound to the surface of the implant in different locations to promote different drug release rates, start and stop times, and overall duration of drug delivery.

The embodiment further includes the one or more growth factors are selected from the group including of transforming growth factors alpha and beta (TGFa, TGFf5n, and TGFp.), insulin-like growth factor (IGF), epidermal growth factor (EGF), fibroblast growth factor (FGF), nerve growth factor (NGF), and platelet-derived growth factor (PDGF).

The embodiment further includes the one or more anti-mitosis drugs are selected from the group including of colchicine, 5-fluorouracil, methotrexate, paclitaxel, and equivalents thereof.

The embodiment further includes the one or more anti-mitosis drugs are covalently bound to an antibody directed towards lens epithelial cells.

The embodiment further includes the intraocular lens being comprised from the group including of polymethylmethacrylate, silicone, acrylate and hydrogels.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top perspective view of an ophthalmic intraocular lens for promoting healing and inhibiting posterior chamber opacification according to an embodiment of the disclosure.

### DESCRIPTION OF THE PREFERRED EMBODIMENT

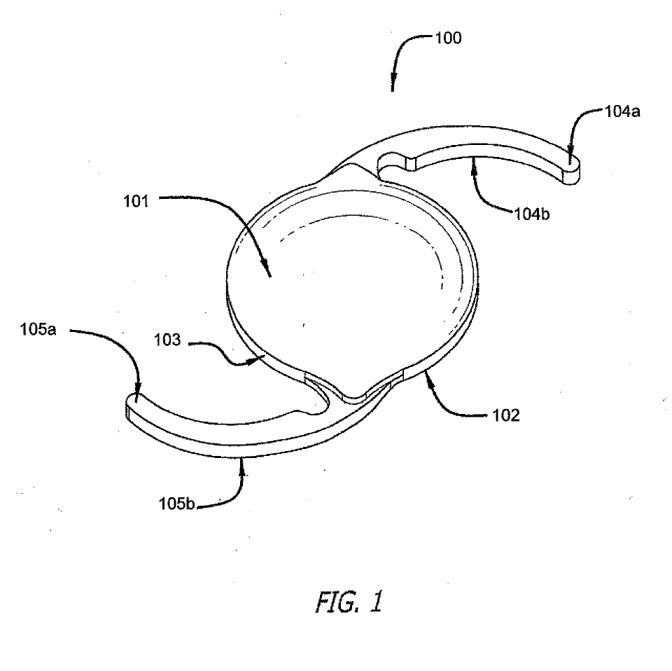

With reference now to the drawings, and in particular to FIG. 1 thereof, a new drug and ophthalmic intraocular lens combination embodying the principles and concepts of an embodiment of the disclosure and generally designated by the reference numeral 100 will be described.

As best illustrated in FIGS. 1 through 6, the ophthalmic intraocular lens for promoting healing and inhibiting posterior chamber opacification 10 generally comprises a combination of mitotic inhibitors to selectively inhibit subcapsular epithelial cell growth and the presence of growth factors that encourages the healing in the cornea that follow cataract and refractive surgery. These materials will be bound and or released from intraocular lens (IOL) surfaces in a controlled fashion. This may be accomplished by the type of bonding to the IOL used or by the location of the materials on the IOL. Generally, the duration should be extended over a period of one to six months.

The present invention allows a mitotic inhibitor, or anti-mitosis drugs (AMDs), effective to inhibit lens epithelial cell growth and fibroblastic proliferation by presenting a cytotoxic agent immediately following cataract or refractive surgery. The IOLs will also contain growth factors. The growth factors, in addition to speeding up the healing process, will speed up the division of the lens epithelial cells and their proliferation. The AMDs are cytotoxic because they block cell mitosis. Therefore, the rate of lens epithelial cell death will actually be increased and enhanced by the presence of growth factors as the growth will be opportunistic for the AMD's. The growth factors and AMDs thusly work in a synergistic fashion. Therefore, the necessary concentration of the AMDs can be lower and the necessary duration needed less than needed otherwise (to kill off the lens epithelial cells), when growth factors are present.

Shown in FIG. 1 is a conventional IOL, though it should be understood that any configuration of IOL may be utilized such a haptic configuration (depicted) or a plate configuration. Intraocular lens 100 has both anterior surface 101 and posterior surface 102. The lens body, and optionally, support members such as are haptics herein, can be made from a material selected from, among others, silicone, polymethylmethacrylate, acrylics, acrylates, polysiloxanes, hydrogel acrylates such as PolyHEMA, PVA, and combinations thereof. It is noted that although the figure depicts the lens with haptics, there are many other support member options and configurations available for intraocular lenses. The lens is concave towards the posterior surface. The posterior surface can be substantially spherical and, after implantation, can reside in contact with the vitreous humor, or posterior bag, in the capsule of the eye. The anterior surface can vary greatly depending on the nature and features of the IOL. IOL 100 also comprises an optic or circumferential edge 103. IOL 100 further comprises support members 104 and 105, which have anterior surfaces 104a and 105a and posterior surfaces 104b and 105b. Support members, such as haptics in this Figure, may be secured to the lens, known as a three piece model, or may be of a continuous material incorporated into the lens (one piece model), optionally, on diametrically opposed sides and aid in centering the lens after implantation.

Recent advances in small-incision cataract surgeries and other foldable IOLs include soft, foldable polymer materials suitable for use in artificial lenses. Conventionally, there are three major categories of these polymers, i.e., hydrogels, silicones, and acrylics. The AMDs and growth factors can be chemically bound to the surfaces of the IOL, or they can be suspended in a coating that is placed onto the surfaces of the IOL. The coating can be erodible or non-erodible. Standard centrifugal or spin coating may be employed to deposit the coatings. The areas of the IOL that are bound or coated may comprise only the haptics, the lens periphery or the optic. However, the whole IOL may be coated or bound with the AMDs and growth factors.

The AMDs and growth factors can be applied to the IOLs together or they can be applied separately. They AMDs and growth factors can also be applied to different locations on the IOL. This way the rate and amounts of each drug that the eye is exposed to can be controlled. For example, it may be beneficial to first expose the eye to growth factors and second to expose the eye to the AMDs. In another example, it may be desirable to expose the eye to short term AMDs (e.g. a day) and growth factors for a longer term (e.g. a week).

To bind the drugs to the surfaces of the IOLs, radiation grafting, plasma polymerization, chemical modification, covalent bonding, non-covalent bonding, combinations thereof or other techniques can be employed. Regarding the AMDs they can be coupled with means to target the cytotoxic agent to particular cell types. Preferably, this is accomplished by covalently coupling AMDs with an antibody, such as anticollagen or anti-lens epithelial cells.

The material used to coat the IOL may be hydrophobic, hydrophilic, or it may have regions of both. Multiple coating materials can be applied to the IOL surface. Each coating applied can be the same material, can each be a different material, or can have several coatings of a single material alternating with one or more coatings of different materials. In order to achieve one or more partial coatings on the IOL surfaces, the areas of the lens that are to remain uncoated may be masked off. Masking may be accomplished by physical or chemical methods. Physical masking techniques include, but are not limited to, covering with plastic, metal, paper or combinations thereof. Chemical masking techniques include, but are not limited to covering with adhesive, soluble coatings, wax or combinations thereof. In addition to simply being able to mask off an area of the lens, e.g., haptics, optics, etc, it is also possible to mask off a portion of a given area. For example, just the tips of the optics may be coated by masking off all of the additional lens assembly. A second different partial coating can be deposited on a pre-coated lens. Different partial coatings can overlap areas that were previously coated or they can be areas that were not previously coated. Several additional different partial coatings can be applied to a lens.

Targeting of the AMDs to specific cells will allow for a much lower concentration of AMDs necessary to be released into the eye's anterior or posterior chamber, thus decreasing the possibility of the patient experiencing harmful side effects. Furthermore, a larger concentration of AMDs can also be used, as needed, with the likelihood of side effects occurring also being lessened due to the targeting of the AMDs specifically to lens epithelial cells (the cells that are proliferating will be targeted). For instance, U.S. Pat. No. 4,432,751 discloses how to prepare monoclonal antibodies against human lens epithelial cells (MAHLEC's). The AMDs can be conjugated to the MAHLEC's and the MAHLEC's will be directed to react only with lens epithelial cells. Therefore, a much lower concentration of AMDs will be needed and the drug will be specifically delivered to the lens epithelial cells. This will also prevent damage by the AMDs on other ocular structures.

AMD-antibody conjugates can be readily synthesized using techniques generally known to those of skilled in the art. The starting materials and reagents used in preparing these conjugates are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Though it should be understood that the invention herein does no lie in the formation of the conjugates.

In one aspect, the AMD is coupled or linked to the antibody via a reactive chemical group on the AMD and/or antibody such that the coupling between the AMD and the antibody results in a covalent bond between the two that is resistant to reducing agents. Reactive chemical groups include, e.g., sulfhydryl groups, amino groups, carboxyl groups, and imidazole groups. The reactive chemical group can be in the hinge region of the antibody. This location reduces or eliminates interference between the antibody/antigen interaction and the AMDs. In one aspect, the AMD can be coupled to the antibody, e.g., via a maleimide group.

The coupling of the AMD to the antibody can also involve an activating agent. Various activating agents that can be used for the coupling reaction include, but are not limited to, 1-ethyl-3-(3-dimethylaminopropyl)carbodiimide (EDC), dicyclohexylcarbodiimide (DCC), N,N′-diisopropyl-carbodiimide (DIP), benzotriazol-1-yl-oxy-tris-(dimethylamino)phosphoniumhexa-fluorophosphate (BOP), hydroxybenzotriazole (HOBt), and N-methylmorpholine (NMM), including mixtures thereof. The coupling reaction can be carried out in solvents such as N-methylpyrrolidone (NMP) or in DMF. In one aspect, conjugation can involve the use of a conjugation kit, such as the Imject Immunogen EDC conjugation kit from Thermo Fisher Scientific In., 3747 N. Meridian Road, Rockford, Ill., 61101. In another aspect, EDC and NMP can be obtained as separate reagents and formulated into a reaction mixture.

The AMD-antibody conjugates in general are specific for an antigen that allows targeting of the conjugates to an abnormally proliferative cell. The growth factors will speed up the proliferation of lens epithelial cells in addition to enhance healing of the eye. Therefore, as previously stated, the growth factors and the AMDs will act in a synergistic fashion. The disclosed AMD-antibody conjugates can also include one or more additional biomolecules. The additional biomolecules can be coupled in the same manner as the AMD's. The biomolecules in an antibody conjugate can be similar as the AMD molecule or different. More specifically, the biomolecules can have the same structure or different structures.

Examples of such AMDs, or anti-mitosis drugs are colchicine, 5-fluorouracil, methotrexate, paclitaxel and known equivalents thereof. Colchicine is a mitosis-inhibiting phenanthrene derivative isolated from Colchicum autumnale. Colchicine arrests mitosis at metaphase by binding to a protein present in microtubules, hence interfering with the structure of the mitotic spindle. The substance has been shown to be a potent inhibitor of lens epithelial cell proliferation and migration. However, colchicine has a low therapeutic index with a lot of potential side effects, including a temporary toxic effect on the optic nerve when used for preventing posterior capsule opacification in primates. 5-fluorouracil is a potent anti mitotic drug affecting the DNA replication and is widely used in the treatment of epithelial tumors. Ruitz et al (Inhibition of posterior capsule opacification by 5-fluorouracil in rabbit; Ophthalmic Res. 22 (1990) 201-208) have also shown that this substance reduces posterior capsule opacification in rabbits. Since 1982 subconjunctival administration of 5-fluorouracil has been utilized in patients at high risk of failure of glaucoma filtering surgery. Although beneficial effects of the substance have been clearly demonstrated, disadvantages have included corneal epithelial defects and other ocular complications. Methotrexate is an antimetabolite and antifolate drug used in the treatment of cancer. Methotrexate competitively inhibits dihydrofolate reductase (DHFR), an enzyme that participates in the tetrahydrofolate synthesis. The affinity of methotrexate for DHFR is about one thousand-fold that of folate for DHFR. Folic acid is needed for the de novo synthesis of the nucleoside thymidine, required for DNA synthesis. Also, folate is needed for purine base synthesis, so all purine synthesis will be inhibited. Methotrexate, therefore, inhibits the synthesis of DNA, RNA, thymidylates, and proteins. Paclitaxel is a mitotic inhibitor used in cancer chemotherapy (also known as Taxol). Paclitaxel is now used to treat patients with lung, ovarian, breast cancer, head and neck cancer, and advanced forms of Kaposi's sarcoma. Paclitaxel is also used for the prevention of restenosis. Paclitaxel stabilizes microtubules and as a result, interferes with the normal breakdown of microtubules during cell division. Together with docetaxel, it forms the drug category of the taxanes.

While it is desirable to prevent cell growth in the posterior chamber, cell growth in the cornea and sclera is critical for wound repair after the cataract surgery. The healing of these wounds can frequently be slow, difficult, and painful with chances of complicating recovery from the postoperative course of surgery. There is, therefore, a need for a method of accelerating ophthalmic wound healing with possible pain reduction or pain elimination. Additionally, the quality of healing of corneal wounds is frequently poor, leading to scarring and other vision-impairing consequences. Therefore, there also is a need for a method that can improve the quality and rate of healing of corneal wounds. It has been found that growth factors may be used to accelerate wound healing and particularly of skin. Growth factors are agents which cause cells to migrate, differentiate, transform, or mature and divide. These factors are polypeptides which can usually be isolated from many different normal and malignant mammalian cell types. Some growth factors can be produced by genetically-engineered microorganisms such as bacteria (Escherichia coli) and yeasts. Among these growth factors are transforming growth factors alpha and beta (TGFa, TGFf5n, and TGFp.), insulin-like growth factor (IGF), epidermal growth factor (EGF), fibroblast growth factor (FGF), nerve growth factor (NGF), and platelet-derived growth factor (PDGF). These are described in U.S. Pat. No. 4,939,135 to Robertson et al., incorporated herein by this reference. In U.S. Pat. No. 5,863,892 to Stern et al., platelet derived growth factor was demonstrated to accelerate corneal anterior stroma healing in mammals in a clinical setting.

Example of a method for attaching AMD and growth factor to an IOL and the test results using such:

### Preparation of Bovine Serum Albumin (BSA) Methotrexate Microspheres

BSA microspheres containing methotrexate (MTX) were prepared by an emulsion technique. An aqueous solution of BSA (12.3-36.6% W/V) was adjusted to pH 10. To 2 ml of BSA solution was added 0.1 g of MTX. This solution was dispersed in a 10-ml solution of Span80 in toluene (10%) by vortexing. This dispersion was stirred and gradually added to 2 ml of glutaraldehyde in distilled water (10%), which was maintained at pH 10. After initial cross-linking (i.e. 20 s), 20 ml of acetone was added with continuous stirring for 10hr. The microspheres formed were washed with acetone five times and with water three times and then dried in a vacuum desiccator to get free-flowing microspheres in a powder form. By varying the drug:polymer ratio, five batches of microspheres were prepared.

TABLE 1
Composition of MTX microspheres of BSA

| I.D. | Drug/Polymer Ratio |
|---|---|
| BSA1 | 1:1 |
| BSA2 | 1:2 |
| BSA3 | 1:3 |
| BSA4 | 1:4 |
| BSA5 | 1:5 |

The microspheres were found to be spherical with quite smooth surfaces.

Drug Content Determination

Five mg of microspheres were accurately weighed and dispersed into 15 ml of KCl-HCl buffer solution of ˜pH 2 by continuous stirring. Then, 10 ml of trypsin solution was added then shaken for 48h in 37° C. Complete dissolution of the matrix components was confirmed by light microscopy. MTX concentration of the resulting solutions was determined.

TABLE 2
Microsphere MTX Content and Entrapment
Efficiency (per 2.5 mg of Microspheres)

| I.D. | MTX Concentration (ug) | Entrapment Efficiency % |
|---|---|---|
| BSA1 | 0.5 | 30 |
| BSA2 | 0.9 | 72 |
| BSA3 | 0.85 | 85 |
| BSA4 | 0.75 | 90 |
| BSA5 | 0.7 | 98 |

The MTX leached from the microspheres was analyzed and compared to pure MTX by melting point, infrared spectroscopy, and by thin layer chromatography and there was no difference indicating the encapsulation process had no detrimental effects on the drug.

In Vitro Drug Release Studies

The drug release studies of the microspheres were carried out for up to three days using paddle dissolution test apparatus. One hundred milliliters of phosphate buffer pH 7.2 was taken as the dissolution medium. Microspheres containing 50 mg-equivalent of MTX were accurately weighed and tied in an isolation cloth.

The dissolution medium was thermally controlled at 37° C. Samples of 1 ml were withdrawn from the dissolution media at suitable time intervals. The sample volume was replaced by an equal volume of fresh medium. The drawn samples were analyzed using a UV spectrophotometer at 303 nm.

TABLE 3
Data for MTX Release from Microspheres

| | MTX Cumulative | MTX Cumulative |
|---|---|---|
| I.D. | Release % (1 day) | Release % (3 days) |
| BSA1 | 30 | 95 |
| BSA2 | 25 | 65 |
| BSA3 | 25 | 55 |
| BSA4 | 25 | 51 |
| BSA5 | 26 | 49 |

### Preparation of Gelatin Epithelial Growth Factor (EGF) Microspheres

10 ml of aqueous solution of acidic gelatin (10 wt. %) was preheated at 45° C. The gelatin solution was added drop wise into 375 ml of olive oil while stirring at 45° C. to yield a water-in-oil emulsion. The emulsion temperature was decreased to 15° C., followed by further continuous stirring for 30 min. After the addition of 100 ml of acetone, the emulsion was further stirred for 1 h. The resulting microspheres were washed with acetone and isopropyl alcohol, centrifuged at 5000 rpm for 5 min, and dried. The non-crosslinked and dried gelatin microspheres (50 mg) were placed in 0.1 wt. % of Tween 80 aqueous solution containing 3 mmol/l of glutaraldehyde (10 ml) and stirred at 4° C. for 15 h to facilitate their crosslinking. After collection by centrifugation at 5000 rpm for 5 min at 4° C., the microspheres were further agitated in 100 ml of 10 mmol/l glycine aqueous solution at 37° C. for 1 hr to block residual aldehyde groups of unreacted glutaraldehyde. The microspheres were finally washed with distilled water, centrifuged at 5000 rpm for 5 min, and freeze-dried.

EGF was incorporated into the gelatin microspheres by dropping 5 mg/ml of EGF solution (20 ml, totally 100 mg of EGF) onto 2 mg of freeze-dried gelatin microspheres, which were then left at room temperature for 1 hr. The solution (20 ml) was completely absorbed into the microspheres during swelling, since the solution volume was less than that theoretically required for the equilibrated swelling of microspheres. These gelatin microspheres have been shown to demonstrate slow extended release of growth factor, e.g., Sakakibara Y, et al., “Toward surgical angiogenesis using slow-released basic fibroblast growth factor” Eur J Cardiothorac Surg 2003; 24:105-111. In our laboratory, these microspheres demonstrated essentially zero order continued release of viable EGF, by enzyme linked immune assay (ELISA) techniques for over 15 days.

### Attaching the MTX and EGF Microspheres to the Test IOLs

The test and control IOLs were 7.0 mm diameter optic single-piece poly(methyl methacrylate) IOLs. Poly(methylmethacrylate) flakes was dissolved in a solvent mixture of acetone and tetrahydrofuran to produce a viscous glue that could be used to glue microspheres to the IOL surfaces and that would evaporate quickly To the test IOLs, the glue was applied carefully to the outer surfaces of the haptics, and the appropriate amount of MTX microspheres and EGF microspheres determined gravimetrically, were fixated by the glue. The control IOLs were used as is.

### Implantation of MTX and EGF Coated (Test) and Control IOLs

The test IOLs consisted of IOLs coated with 0.01 grams of BAS4 MTX microspheres and 0.001 grams of EGF microspheres attached to the IOL haptics prepared as described above. The control IOLs were used as is. Ten eyes of 5 New Zealand albino rabbits were utilized in the study. Extracapsular cataract extraction and posterior chamber intraocular lens (IOL) implantation were performed in all cases using general anesthesia. A test IOL was implanted in one eye and a control IOL was implanted in the opposite eye which served as an operational control. Postoperatively, atropine 1%, tobramycin, and fluorometholone eye drops were instilled twice daily for one week.

To determine the effects of the MTX and EGF on the remnant epithelial cells, two rabbits were sacrificed at three weeks postoperatively despite the lack of any opacification on the posterior capsule wall on either the control or the test eyes. The capsules were isolated for electron microscopy. The test samples revealed a homogeneous population of spindle shaped cells in the angles between the posterior and anterior capsules. Any remnant cells in the test group had non-mitotic, contorted interphase nuclei, atrophic mitochondria, and bloated rough endoplasmic reticulum.

The remaining three rabbits were euthanized at 3 months postoperatively. The eyes were harvested for histopathology. The control eyes were completely opacified by the proliferating lens epithelial cells. The test eyes had a clear anterior and posterior capsule devoid of any lens epithelial lens remnant cells. No ocular toxicity, e.g., corneal thickness, retinal damage, corneal healing etc, was observable in either the control or test population.

In use, the IOL will be utilized in a conventionally known manner and relates to different kinds of intraocular lenses for implantation in an aphakic or phakic eye. However, the combination of the AMDs and growth hormones positioned on the IOL will inhibit preventing posterior chamber opacification (PCO) while promoting the healing of the eye. PCO is also regarded as secondary cataract, which a long term complication after extracapsular cataract extraction with intraocular lens (IOL) implantation. While the construction of the IOLs and the implantation techniques for such have both improved, this problem has not been resolved. However, the combination of AMDs and growth hormones provided to the capsular bag by the IOL, over an extended period of time will inhibit epithelial cell growth while the presence of growth factors that encourages the healing in the cornea that follow cataract and refractive surgery.

It is often desirable to maintain and ship the IOL in an aqueous medium. For example, hydrogel lenses often take days to hydrate and stabilize after their manufacture. Many AMDs and growth factors are unstable when stored in an aqueous medium for extended periods. Therefore, packaging a drug treated IOL necessitates that the attached drugs are not exposed to an aqueous medium for extended periods. One technique to package a hydrated IOL that can deliver drugs and other compounds is to utilize a blister package such as described in US 2005/0199648 by Schiller et al. For example in a triple compartment blister package the hydrated IOL, AMDs, and growth factors can be placed into separate compartments. The AMDs and growth factors can thusly be kept dry and stabilized. Hours or days prior to implantation, the compartments can be mixed together to allow the AMDs and growth factors to be attached to the surfaces of the IOL while still being maintained in a sterile package. After the appropriate incubation period the treated IOL can then be implanted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure.

I claim:

1. An eye implant assembly comprising:
an intraocular lens; and
a composition being bonded to or coated on said intraocular lens, said composition including one or more growth factors and one or more anti-mitosis drugs.

2. The assembly according to claim 1, wherein said one or more growth factors and said one or more anti-mitosis drugs are covalently, non-covalently, or a combination thereof bound to the surface of said implant using wet chemical, photochemical, plasma, gamma irradiation or polymerization techniques.

3. The assembly according to claim 1, wherein said one or more growth factors and said one or more anti-mitosis drugs are bound to the surface of said implant in different locations to promote different drug release rates, start and stop times, and overall duration of drug delivery.

4. The assembly according to claim 1, wherein said one or more growth factors are selected from the group consisting of transforming growth factors alpha and beta (TGFa, TGFf5n, and TGFp.,), insulin-like growth factor (IGF), epidermal growth factor (EGF), fibroblast growth factor (FGF), nerve growth factor (NGF), and platelet-derived growth factor (PDGF).

5. The assembly according to claim 1, wherein said one or more anti-mitosis drugs are selected from the group consisting of colchicine, 5-fluorouracil, methotrexate, paclitaxel, and equivalents thereof.

6. The assembly according to claim 1, wherein said one or more anti-mitosis drugs are covalently bound to an antibody directed towards lens epithelial cells.

7. The assembly according to claim 1, wherein said intraocular lens is comprised from the group consisting of polymethylmethacrylate, silicone, acrylate and hydrogels.

8. A method of implanting an implant within an eye, said method comprising the steps:
creating an incision in an eye;
providing an intraocular lens, said intraocular lens including a composition being bonded thereto or coated thereon, said composition including one or more growth factors and one or more anti-mitosis drugs; and
implanting said intraocular lens in said eye through said incision.

9. The method according to claim 8, wherein said step of providing said intraocular lens further includes said one or more growth factors and said one or more anti-mitosis drugs are covalently, non-covalently, or a combination thereof bound to the surface of said implant using wet chemical, photochemical, plasma, gamma irradiation or polymerization techniques.

10. The method according to claim 8, wherein said step of providing said intraocular lens further includes wherein said one or more growth factors and said one or more anti-mitosis drugs are bound to the surface of said implant in different locations to promote different drug release rates, start and stop times, and overall duration of drug delivery.

11. The method according to claim 8, wherein said step of providing said intraocular lens further includes wherein said one or more growth factors are selected from the group consisting of transforming growth factors alpha and beta (TGFa, TGFf5n, and TGFp.), insulin-like growth factor (IGF), epidermal growth factor (EGF), fibroblast growth factor (FGF), nerve growth factor (NGF), and platelet-derived growth factor (PDGF).

12. The method according to claim 8, wherein said step of providing said intraocular lens further includes wherein said one or more anti-mitosis drugs are selected from the group consisting of colchicine, 5-fluorouracil, methotrexate, paclitaxel, and equivalents thereof.

13. The method according to claim 8, wherein said step of providing said intraocular lens further includes wherein said one or more anti-mitosis drugs are covalently bound to an antibody directed towards lens epithelial cells.

14. The method according to claim 8, wherein said step of providing said intraocular lens further includes wherein said intraocular lens is comprised from the group consisting of polymethylmethacrylate, silicone, acrylate and hydrogels.
